(19) United States
(12) Patent Application Publication
(10) Pub. No.: US 2020/0068797 A1
(43) Pub. Date: Mar. 5, 2020

(54) METHOD AND SYSTEM FOR ESTIMATING EFFECTIVE CROP NITROGEN APPLICATIONS

(71) Applicant: Farmers Edge Inc., Winnipeg (CA)

(72) Inventors: Solomon Muleta Folle, Spring Lake Park, MN (US); Jose Adolfo Hernandez, Saint Paul, MN (US); Mahmudul Hasan, Calgary (CA)

(73) Assignee: Farmers Edge Inc., Winnipeg (CA)

(21) Appl. No.: 16/532,219

(22) Filed: Aug. 5, 2019

Related U.S. Application Data
(60) Provisional application No. 62/725,934, filed on Aug. 31, 2018.

(51) Int. Cl. A01C 21/00; G01N 33/24; G06Q 50/02; G06F 17/50

(52) U.S. Cl. CPC A01C 21/007; G01N 33/24; G01N 2033/245; G06F 17/5009; G06Q 50/02

(57) ABSTRACT

A method for effective nitrogen application may include acquiring imagery of an agricultural field and delineating a plurality of management zones within the agricultural field using the imagery of the agricultural field. For each of the management zones within the agricultural field, the method may include receiving soil characteristics at a computing device, the soil characteristics derived from physical soil samples within the management zones. For each of the management zones within the agricultural field, the method may include receiving weather data at the computing device, management practice information and crop cultivar identification at the computing device. The model simulates effects of in-season nitrogen applications on crop yields within each of the management zones.

### PRIORITY STATEMENT

This application claims priority to U.S. Provisional Application No. 62/725,934, entitled, “Plant Models for Estimating Effective Crop Nitrogen Applications”, filed Aug. 31, 2018, hereby incorporated by reference in its entirety.

### TECHNICAL FIELD

The present invention relates to application of crop nitrogen fertilizer to crops. More particularly, but not exclusively, the present invention relates to a method and system used for achieving maximum economic results for crop nitrogen fertilizer applications using crop models and non-linear parameter estimation.

### BACKGROUND

One of the mandates of precision agriculture is to optimize the amount of fertilizer used in crop production. This can be achieved through variable-rate fertilizer applications, where the amount of fertilizer applied varies across a field, according to the needs at each particular location. What determines the fertilizer needs at any given point in the field is governed by the crop nutrient demand and the availability of such nutrients in the soil solution and their diffusion rates through the soil. Effective nutrient recommendations can be made through analysis of the overall interaction of crop genetics, soils, and landscape features such as topography, soil texture/water holding capacity, soil nutrient levels, weather, and agronomic management practices.

An adequate supply of nitrogen in cereal crops is critical for high yields and economic profitability (Sawyer, 2015). Optimal nitrogen fertilization not only contributes to economic success, but also to the minimization of nitrate transport to surface and subsurface water, which can be a great environmental concern (Hernandez and Mulla, 2008). A strategy based on the maximum economic return to nitrogen fertilizer allows farmers to consider changes in the cost of nitrogen and the price of their crop, while lessening the chance of negative environmental impact.

Current approaches used to predict the application of optimal nitrogen rates include yield-goal based nitrogen recommendations, pre-plant, and pre-side dress soil nitrate tests, soil nitrogen tests, crop canopy sensing (NDVI), chlorophyll meters, and maximum return to nitrogen (MRTN) (Puntel et al., 2016). None of these strategies fully meets the needs of the industry as they are fraught with various drawbacks such as high costs, unreliability, or negative environmental impacts (Puntel et al., 2016).

It remains a challenge within the industry to manage nitrogen fertilizer application in such a way that the crop reaches its economic growth potential and nitrogen loss to the environment is minimized. Estimating the maximum economic return to nitrogen fertilizer allows for the consideration of nitrogen cost and crop price. The goal is to derive the most economic yield, which is not necessarily the maximum yield.

### SUMMARY

Therefore, it is a primary object, feature, or advantage of the present invention to improve over the state of the art.

It is a further object, feature, or advantage to manage nitrogen fertilizer application in such a way that the crop reaches its economic growth potential and nitrogen loss to the environment is minimized.

A still further object, feature, or advantage of the present invention is to use a simulation model rather than a field trial for predictive measures.

Another object, feature, or advantage of the present invention is to account for all stages of a crop including planting, vegetative growth, reproductive growth, and harvest.

It is a further object, feature, or advantage of the present invention to provide a crop model which uses pre-plant nitrogen levels using simulated data.

It is a still further object, feature, or advantage to provide daily, dynamic nitrogen availability information to assist growers with selecting the correct rate and timing for side dressing nitrogen to accomplish yield goals by production zone, resulting in maximum economic returns.

It is another object, feature, or advantage to use real-time, field-centric data monitored at a fine spatial scale to assist in managing nitrogen fertilizer application management.

It is yet another object, feature, or advantage to provide a number of deliverables to a grower such as, but not limited to side-dress nitrogen recommendations to parcels of land (zones) within a grower's field, predicted yield, dates to the six-leaf growth stage, and the predicted major nitrogen balance components that include nitrogen losses and gains, nitrogen uptake, and soil nitrate level.

A still further object, feature, or advantage is to integrate NDVI-derived production zones and field-centric data such as soil type and zone soil test results, current weather from on-farm weather stations, crop cultivars, genetic coefficients, and agronomic operations including planting density, planting depth, row spacing, and manure and fertilizer input data.

Another object, feature, or advantage is to select the correct rate and timing for side dressing nitrogen needs, helping growers accomplish their yield goals by production zone.

One or more of these and/or other objects, features, or advantages will become apparent from the specification and claims that follow. It is to be understood that different embodiments may have different objects, features, or advantages and therefore the present invention is not to be limited by or to any object, feature, or advantage listed herein.

According to one aspect of the present invention, a solution for effective crop nitrogen applications is provided which uses a crop model and non-linear equations. An estimate of maximum economic return to nitrogen is calculated by fitting a regression model with crop yield and applied nitrogen as the variables. This disclosure defines the methods and systems used in the estimation of maximum economic return to nitrogen fertilizer in a crop such as corn using crop models and non-linear parameter estimation. The disclosed method uses a process-based model to simulate corn yields at various added nitrogen applications. A zone-specific nitrogen modeling tool, driven by field-centric data, is implemented for effective in-season nitrogen recommendations for corn. Local weather and soil bio-geochemical properties, combined with planted corn cultivars or hybrid genetic coefficients, are incorporated into the simulation processes. Calculations of economic nitrogen rates are made using linear-plateau and quadratic-plateau equations.

According to another aspect of the present invention a method for effective nitrogen application is provided. The method may include acquiring imagery of an agricultural field and delineating a plurality of management zones within the agricultural field using the imagery of the agricultural field. For each of the management zones within the agricultural field, the method may include receiving soil characteristics at a computing device, the soil characteristics derived from physical soil samples within the management zones. For each of the management zones within the agricultural field, the method may include receiving weather data at the computing device. For each of the management zones, the method may include receiving management practice information and crop cultivar identification at the computing device. The method may further include applying a crop model implemented by instructions stored on a computer readable medium and executed on the computing device to simulate effects of in-season nitrogen applications on crop yields within each of the management zones, wherein the crop model is parameterized with the soil characteristics, the weather data, the management practice information, and the crop cultivar identification in order to provide in-season nitrogen recommendations for the crop. The method may further include updating the crop model a plurality of times during the growing season. The updating of the crop model may include providing updated weather data to the crop model. The updating of the crop model may occur on a periodic basis such as a daily basis during the growing season. The soil characteristics may include soil texture, organic matter, pH, cation exchange capacity (CEC), and soil nitrogen content. The crop model may use linear-plateau and quadratic-plateau equations to calculate effects of different nitrogen rates. The crop model may simulate crop yields at a plurality of added nitrogen rates. The crop model may be further parameterized with a crop price and a nitrogen fertilizer cost. The crop model may determine a net return for each of a plurality of added nitrogen application rates. The crop model may determine an estimate of a maximum economic return for nitrogen fertilizer using a quadratic-plateau model.

According to another aspect, a system for effective nitrogen application within an agricultural field during a growing season is provided. The system includes a computing environment including at least one computer-readable storage medium having program instructions stored therein and a computer processor operable to execute the program instructions to apply a crop model. The crop model simulates effects of in-season nitrogen applications on crop yields within each of a plurality of management zones within the agricultural field. The crop model is parameterized with soil characteristics obtained from soil samples within the plurality of management zones, weather data including weather data collected during the growing season, management practice information, and crop cultivar identification in order to provide in-season nitrogen recommendations for the crop. The soil characteristics may include soil texture, organic matter, pH, cation exchange capacity (CEC), and soil nitrogen content. The crop model may use linear-plateau and quadratic-plateau equations to calculate effects of different nitrogen rates. The crop model may simulate crop yields at a plurality of added nitrogen application rates. The crop model may be further parameterized with a crop price and a nitrogen fertilizer cost. The crop model may determine a net return for each of a plurality of added nitrogen application rates and/or an estimate of a maximum economic return for nitrogen fertilizer using a quadratic-plateau model.

### BRIEF DESCRIPTION OF THE DRAWINGS

The disclosed embodiments have other advantages and features which, may be garnered in part by study of the accompanying figures (or drawings). A brief introduction of the figures, referred to in numerals is below.

FIG. 1 illustrates a system environment for determination of maximum economic return to nitrogen, according to one example embodiment.

FIG. 2 shows nitrogen dynamics routines for nitrogen balance.

FIG. 3 shows an example of system environment deliverables to the grower.

FIG. 4 is an example of maximum economic return to nitrogen fertilizer. The inflection point, marked maximum, is where the return becomes flat and consistent.

FIG. 5 illustrates a corn variety defined by six genetic coefficients and ecotype codes.

FIG. 6 shows an example of water balance parameters.

FIG. 7 shows an example of the distribution of average monthly precipitation.

FIG. 8 illustrates non-linear equation estimation.

FIG. 9 illustrates an example of the methodology.

FIG. 10 illustrates an example of a computing environment.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the disclosed principles. It is noted that wherever practicable, similar or like reference numbers may be used in the figures and may indicate similar or like functionality. The figures depict embodiments of the disclosed system (or method) for purposes of illustration only.

### Overview

A typical ex post approach to predicting optimal nitrogen requirements is to run actual field experiments followed by a simulation. A crop is planted, and different nitrogen rates are applied pre-plant, in-season, or as side-dress in the fall. Once the harvest is complete, a post-mortem analysis is performed on the collected data and a response curve calculated. A simulation is run in preparation for the following crop season, incorporating the available factors affecting nitrogen application and crop yield.

The new approach in this method determines pre-plant nitrogen levels using simulated data. The crop model used in this method integrates information collected by agronomists on soil type, current weather, crop growth and development (phenology) and crop yield that are vital for model calibration and validation. Additionally, different cultivars and different relative plant maturities may be accounted for within the model. All these factors may be combined in the simulation. Nitrogen applications can then be optimized to reach yield targets and increase profitability.

Described herein is a method and system for achieving maximum economic results for crop nitrogen fertilizer applications using crop models and non-linear parameter estimation. In one embodiment, a model is described that provides daily, dynamic nitrogen availability information to assist growers with selecting the correct rate and timing for side dressing nitrogen to accomplish yield goals by production zone, resulting in maximum economic returns. A detailed description of the processes and algorithms utilized in this system follows below, including specific examples.

### System Environment

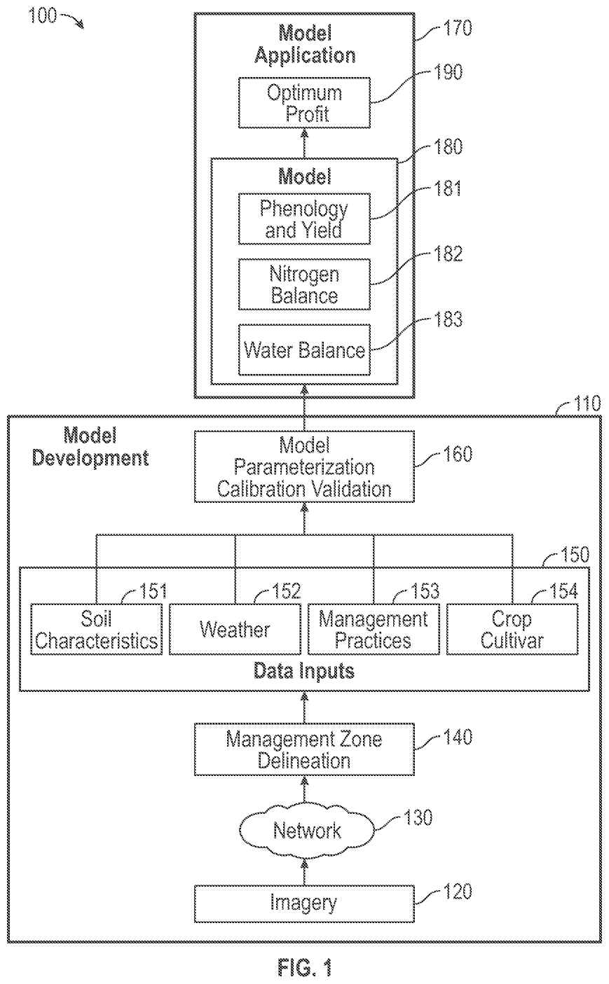

FIG. 1 illustrates a system environment 100 for determination of maximum economic return to nitrogen, according to one example embodiment. Within the system environment 100 is a model development system 110 and a model application system 170.

In the model development system 110, process-based mechanistic crop modules have been used to simulate field-scale nitrogen dynamics and their interaction with spatial and temporal variability of soils, climate, crop cultivar, and applied agronomic management practices. Crop models used here may be acquired from tested and trusted publicly available sources, which are known to a person skilled in the technical field. The main engine behind the model may be, for example, DSSAT.

Central to the model is the use of real-time, field-centric data monitored at a fine spatial scale. A network of high density, micro-climate monitoring equipment installed close to farm fields is used to monitor real-time precipitation, temperature, and wind speed. Soil samples are collected on site for the analysis of soil carbon, pH, cation exchange capacity, and soil nitrogen content. The field specific weather data and soil characteristics are combined with client provided crop cultivar information and farm management practices and then used as input parameters for the development of the model.

Imagery 120 is remotely sensed data in an observed image. Herein, an observed image is an image or photograph of an agricultural field taken from a remote sensing platform (e.g., an airplane, satellite, or drone). Imagery data includes data from satellite images (e.g., PlanetScope™, RapidEye™, PlanetScope Mission 2™, SkySat™, LandSat™ 7, 8, and Sentinel™). The observed image is a raster dataset composed of pixels with each pixel having a pixel value. Pixel values in an observed image may represent some ground characteristic such as, for example, a plant, a field, or a structure. The characteristics and/or objects represented by the pixels may be indicative of the crop conditions within an agricultural field in the image. Remote sensing measurements of the crop leaf reflectance of the electromagnetic spectrum are represented by the normalized difference vegetation index (NDVI) to provide a crop greenness coefficient or index.

Imagery 120 is transmitted to the management zone delineation process 140 via a network 130. The network 130 is typically the Internet but can be any network(s) including but not limited to a LAN, a MAN, a WAN, a mobile wired or wireless network, a private network, a virtual private network, or a combination thereof.

The management zone delineation process 140 couples high resolution soils data with imagery 120 to establish innovative production farm management zones. These zones are considered modeling units whereby each zone has a unique soil physical and chemical characteristic, slope, weather, management practice, and production potential for which a distinct, real time, site specific, in-season nitrogen recommendation is made.

A data input system 150 is a system which provides field-centric data from an agricultural field, monitored at a fine spatial scale. In an embodiment, soil characteristics 151 are derived from zone-based soil samples collected and analyzed for texture, organic matter, pH, CEC, and soil nitrogen content. Soil variability exists within fields and this variability causes differences in the productive capability of soils within the same field. Areas within the same field, sometimes in close proximity, show large differences in soil composition. This can greatly affect the amount of organic matter, which can cause a difference in the amount of mineralized nitrogen available for plant growth. It can also affect the water holding capacity of the soil which leads to yield.

Weather is obtained from on-farm weather stations 152, which are used to monitor real-time precipitation, temperature, solar radiation, soil moisture, soil temperature, and wind speed. The most accurate field-level weather information comes from measuring it directly in the field using weather stations. On-farm weather stations report temperature, humidity, dew point, wind speed/direction, barometric pressure and rain, which includes daily and hourly precipitation. Heavy precipitation events can cause yield loss as they increase the loss of nitrogen from the soil through leaching and volatilization. Therefore, early season weather monitoring is important to yield management. A ten-day weather forecast used to model forward is centered around weather stations. Historic weather data and forecast information is supplied by The Weather Company, which can provide 30 years of historical data. High density weather data are used as inputs to the model.

The client provides farm management practices 153 and crop cultivar 154. Farm management practices are under different geographic settings and environmental conditions. Management practices are used as input parameters for the process-based dynamic crop simulation models. Management practices including planting data, fertilizer application amounts, and dates can be changed in the model.

The final phase of model development 110 is model parameterization, calibration, and validation 160. The model uses a hierarchical method that involves input parameter screening and spatial parameterization through scrutiny of parameter specifications and parameter estimation approaches. Known parameters are based on research data or empirical observations.

Crops behave differently in different agroclimatic regions and, therefore, developed crop growth and nitrogen dynamic modules are extensively calibrated and validated, on a daily time step, under diverse geography, environmental settings, and management scenarios. Comparison of the simulated outputs against measured crop growth and development stages, leaf area index, grain yield, nitrogen uptake and nitrogen losses prove the model's high simulation efficiency.

Validation of the simulated crop phenology, grain yield, soil moisture/water balance, nitrogen balance, and crop nitrogen requirements at different development stages of the crop over the growing season demonstrate the model's ability to efficiently assist growers with nitrogen management decisions that result in optimum profit. At this step, the model is validated to ensure that it is performing properly and producing correct results.

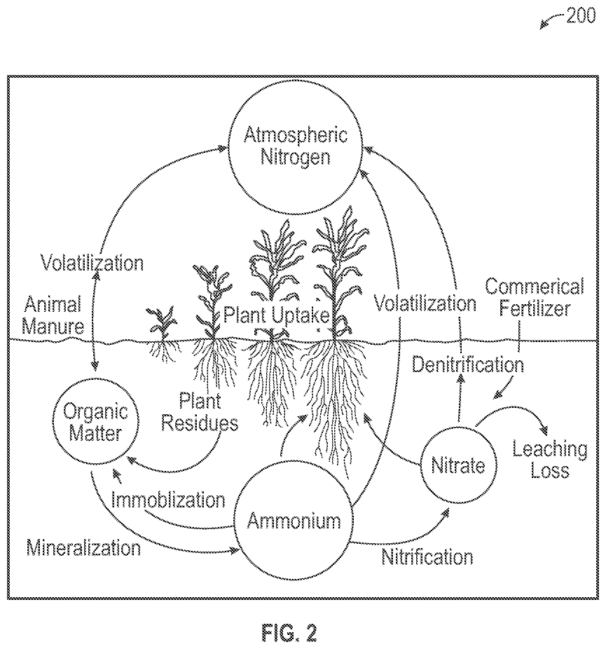

The setup and configuration of model application 170 for nitrogen recommendation solutions simulates the farm management zones nitrogen cycle and harmonizes the soil nitrogen availability and plant nitrogen demand to achieve optimum yield and, therefore, optimum profit 190. The model incorporates sub-routines for water balance 183 that effectively track the hydrologic processes and availability of sufficient soil moisture for crops grown. FIG. 2 shows nitrogen dynamics routines 200 for nitrogen balance 182. These routines are designed to effectively simulate the availability of nitrogen in the different soil pools, crop nitrogen uptake, nitrogen losses via surface runoff and leaching, additions to the soil from mineralization of organic matter, crop residue and animal manure, and crop development and effects of nitrogen deficiency on crop growth processes.

Phenology and yield 181 are the result of specific crop varieties and locally calibrated genetic characteristics of a crop. Previous year crop yield for each zone is used to help calculate residue from the previous growing season. This information is used to simulate fresh organic residue decomposition, which adds to the available nitrogen.

Management operations including planting data, fertilizer application amounts, and fertilizing dates can be changed within the model. Ultimately, the simulation outputs are used by the grower to make a decision on when and how much nitrogen fertilizer they need to apply to a given farm field under corn crop so that optimum grain yield may be secured and thus optimum profit 190.

### Detailed Description of Applied Model

Growers must supply supplemental nutrients to crops to ensure optimal growth and to maximize profit. These supplemental nutrients, nitrogen in particular, come in numerous forms comprising mineral fertilizers, animal manures, green manures, and legumes. Many different physical and chemical forms of commercial fertilizers are available as solids, liquids, or gases. Each physical form has its own uses and limitations, which provide the basis for selecting the best material for the job. Fertilizer cost and economic yield are important factors in determining optimum profit 190.

The model 180 used in this method consists of electronic digitally stored executable instructions and data values associated with one another. This model 180 can receive and respond to digital calls to yield output values for computer-implemented recommendations generated by data modeling and analytics. As data is collected for model 180, it is processed to obtain values that drive analytics and decision-making functions. Functions created may be shared and/or distributed to authorized users and subscribers. The processing of data occurs in both model development 110 and model application 170, with the resulting processed data pushed down to authorized users or subscribers, for example, in the form of a custom report generated by the system environment 100.

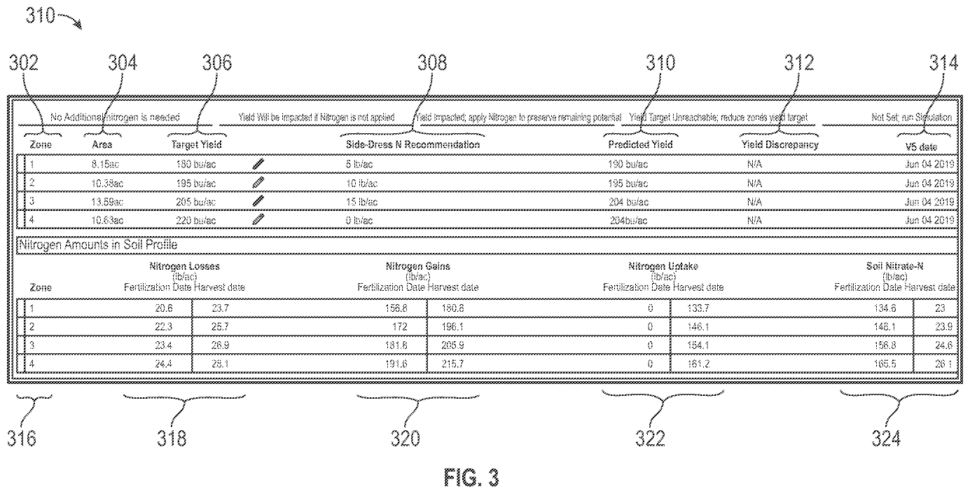

Ultimately, the system environment 100 provides several deliverables to a grower. These include side-dress nitrogen recommendations to parcels of land (zones) within a grower's field, predicted yield, dates to the six-leaf growth stage, and the predicted major nitrogen balance components that include nitrogen losses and gains, nitrogen uptake, and soil nitrate level. An example of the deliverables is shown in FIG. 3.

As shown in FIG. 3, the representative deliverable 300 illustrates zone identifiers 302. Associated with each zone is an area 304 which may be represented in acres and indicates the size of the zone. Target yields 306 are provided for each of the zones. The target yields may be shown in bushels per acre. Side dress nitrogen recommendations 308 are also provided. Predicted yields 310 are also provided. Yield discrepancies 312 between the target yield and the predicted yield may be provided where applicable. A V6 growth stage date 314 may also be provided.

Nitrogen amounts in the soil profile may also be provided. For each zone 316, nitrogen losses 318 may be provided for the fertilization date as well as the harvest date. Nitrogen gains 320 may also be provided for the fertilization date as well as the harvest date. Nitrogen uptake 322 may also be provided for the fertilization date as well as the harvest data. Soil nitrate levels 324 may also be provided for the fertilization date and the harvest date.

Returning to FIG. 1, model application 170 occurs when the model 180 used in this method integrates NDVI-derived production zones and field-centric information including the following: soil type and zone soil test results, current weather from on-farm weather stations, crop cultivars, genetic coefficients, and agronomic operations including planting density, planting depth, row spacing, and manure and fertilizer input data. Information is collected by agronomists and combined in the simulation with updates occurring automatically on a daily basis. Nitrogen applications are then optimized to efficiently reach productive yield targets and increase profitability, while reducing environmental impacts through nutrient stewardship.

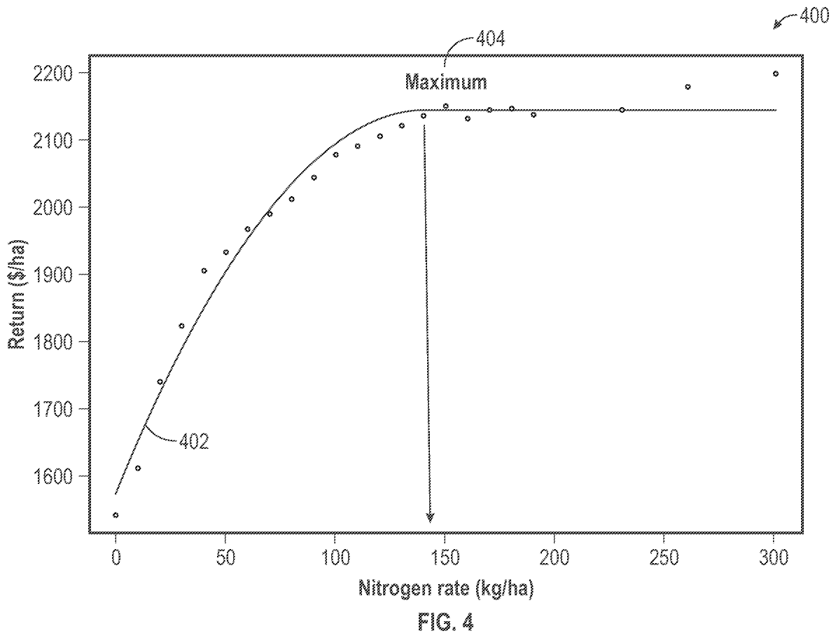

FIG. 4 depicts an example 400 of the economic maximum, whereby the inflection point identifies when the return becomes flat and consistent, indicating that further nitrogen does not provide economic benefit. Additional nitrogen applications will not show an economic response given conditions to date and expected future conditions. Growers are able to use this information to select the correct rate and timing for side dressing nitrogen needs, helping them accomplish their yield goals by production zone.

With model 180, nitrogen recommendations are derived from the establishment of nitrogen requirements, or nitrogen balance 182, and the phenology and yield 181 response of the crop to nitrogen. Water balance 183 is also taken into consideration. Work proceeds using incremental nitrogen application rates that avoid crop stress from the lack of nitrogen. The model 180 calculates the balance between nitrogen added to the field and the amount removed per hectare of field.

This invention is unique in that it uses a simulation model 180, rather than a field trial, for predictive measures. Model 180 is a zone-specific nitrogen management tool for all in-season applications. Soil input information for model 180 is not an interpolated estimation but is derived from empirical data of physical soil samples used to quantify soil nitrogen. With this technique, accurate input information is sent to the model 180, resulting in accurate output, thereby reducing uncertainty in the simulation. In this scheme, the crop is planted and model 180 is subsequently run to predict nitrogen deficiency and determine if an application of nitrogen is required. As shown in FIG. 4, there is a curve 402 fit to the data having a maximum 404. All stages of a crop including planting, vegetative growth, reproductive growth, and harvest are accounted for in model 180.

With this method, the complexity of nitrogen management and soil variability is recognized. Nitrogen is very mobile in the soil and its movement depends on weather and soil properties as they vary in space and time. Heavy precipitation events increase nitrogen loss; therefore, tracking early season weather is important for yield management. During spring rains, nitrogen changes are predictable, making this a valuable time for fertilizer intervention. Model 180 determines how much of the fall and early spring nitrogen application was lost and how much was gained through mineralization. It predicts if there is sufficient nitrogen in the soil system to maximize yield and if additional nitrogen applications would show response given conditions to date and expected future conditions.

Overall nitrogen balance 182 or mass balance is an important consideration necessary for understanding options for management improvements and the mitigation of environmental impacts of nitrogen. Nitrogen balance 182 in each zone is calculated at two different levels: the annual nitrogen balance, and the post-harvest (October samples) and pre-plant (April samples) measures. A determination must be made as to whether there is sufficient nitrogen in the soil to maximize yield or if additional nitrogen is necessary. Zones that do not require nitrogen application have relatively high amounts of soil nitrate post harvest and pre plant. Other zones may have unreachable yield targets or be in a warning state with inadequate levels of soil nitrate available. Soil nitrate simulation outputs from the model 180 have very high accuracy and can be used as predictors to supplement expensive soil lab analyses.

Pre-plant nitrogen levels are obtained from the simulated data. Total nitrogen percent is calculated by extracting the percent organic matter from an available soil test. If a soil test is not available, high resolution digital soil data is obtained from the US Soil Survey Geographic Database (SSURGO), which provide access to soil sampling data that includes physical and chemical records for various types of soil. In Canada, the National Soil Database (NSDB) is utilized. The percent organic carbon is calculated from the organic matter and then total nitrogen is represented by approximately 10% of the organic carbon.

The simulator will calculate the nitrogen status based off all the variables entered into the model to date and will also identify if there is any leaching or mineralization. Soil nitrogen mineralization is a continual process whereby plant-available nitrogen is produced before and after planting. This is tightly regulated by the demand for organic carbon and nitrogen from soil microbes. The rate of mineralization varies with soil temperature, water content, soil type, organic matter, crop residues, and pH. The process occurs more slowly in acidic soils. More mineralization may occur when no nitrogen fertilizer is applied. The amount of nitrogen mineralized has a positive relationship with soil organic carbon. It is not the only predictor and it is not always a good predictor. Nitrogen mineralization in model 180 is based on the Crop Environment Resource Synthesis (CERES) crop growth model. An accurate prediction of organic matter decomposition and release of nitrogen through mineralization requires adequate quantification of fresh organic residue left on the field from the previous crop in the preceding growing season.

The predicted corn yield is on a dry matter basis and required adjustment for its moisture content. For this procedure, the national standard of 15.5% grain moisture content is used to calculate the moist weight of the grain. This approach has a limitation as it does not consider the fact that moisture content of a given hybrid varies from field-to-field, from environmental impact, or year-to-year.

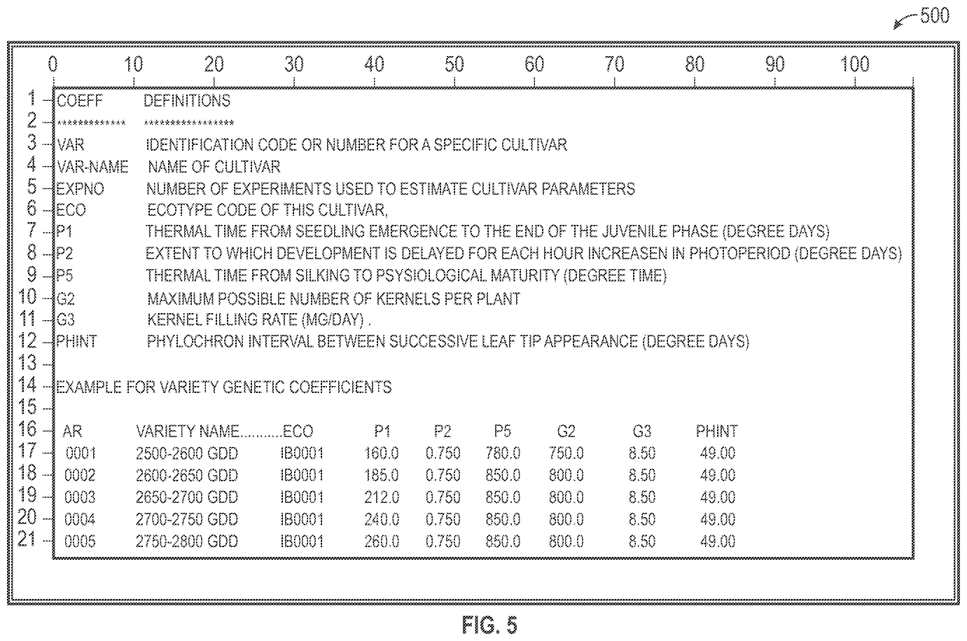

Phenology and yield 181 in model 180 are the result of specific crop varieties and locally calibrated genetic characteristics. Crop and variety information may be retrieved from a seed variety database. There are several different chemical companies that provide hybrid seeds and the genetic engineering services. As required by governance, every bag of seed must have a seed variety number on it and the seed varieties used for farming operations are tracked. In this invention, co-characterization may be implemented with crop database information and actual field measurements of genetic crop characteristics. Commercial verification sites (CVS) and super verification sites (SVS) are used for calibration of crop genetic characteristics that determine growth and development (phenology) and yield. Each corn variety may be defined by six genetic coefficients and ecotype codes. An example is shown in FIG. 5 which includes a screen display 500 showing crop genetic coefficients.

Plant life cycle events are always influenced by seasonal and interannual variations in climate, as well as environmental factors such as topography. The six-leaf stage (V6) is often the choice of growers for side-dress nitrogen applications and the date for V6 is affected by seasonal weather factors. Variability in crop response to nitrogen may be accounted for by differences in soils, climatic conditions, hybrids, planting dates, planting density, planting depth, tillage, and other management aspects.

Simulated yield is based on target yield. Pre-plant estimated yield must be realistic and close to actual harvest. Predicted yield is the result of a number of factors: 1) field-centric and modeled future weather for the zone, 2) nitrogen from field-centric soil tests and soils information, 3) all applications that have been added to the zone, 4) soil nitrogen gains and losses from the zone, and 4) the addition of the recommended side dress rate from the model.

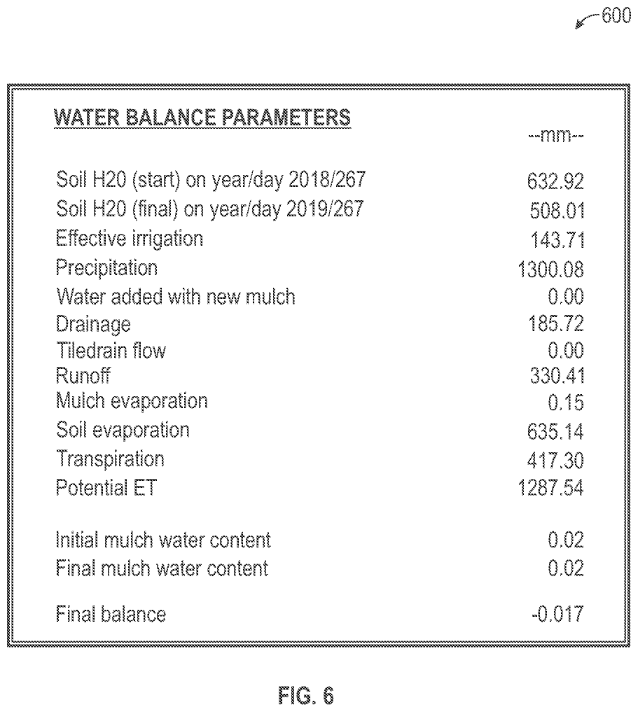

Another factor of model 180 for nitrogen recommendations is water balance 183, which is tied to weather, crop growth stages, and evapotranspiration. Ten years of historical weather for long-term forecasting, growth stages, and growing degree stages from growth stages may be used to calculate evapotranspiration. In addition, the growing season climatic index, which relies on 30 years of historical weather for long-term forecasting, may be used. An example of water balance parameters 600 is shown is FIG. 6.

An additional feature, which sets this invention apart from the prior art, is its ability to account for current weather in the model. As methods to estimate the maximum return to nitrogen applications are commonly determined ex post, historical weather data is utilized. The model in this method is driven by current weather data from weather stations located within five kilometers of the field making it a unique and highly accurate model. An estimation of future weather for the remaining days of the season is required and is assumed to be normal or average weather conditions. Predictions of average weather are based on a normal rainfall year selected from the previous ten years of historical weather data. All weather data is represented, including rainfall, maximum and minimum temperatures, solar radiation, wind speed, relative humidity, and dew point. A step-by-step process is outlined below to identify a normal year using 10 years of historical data.

Step 1: take the last 10 years of daily weather data and calculate the average monthly and annual precipitation. An example is shown below.

| | | | | | | | | | | | | | Annual |
|---|---|---|---|---|---|---|---|---|---|---|---|---|---|
| | | | | | | | | | | | | | PRCP, |
| Year | January | February | March | April | May | June | July | August | September | October | November | December | inches |
| 2007 | 0.86 | 1.55 | 2.89 | 1.7 | 3.13 | 2.87 | 2.66 | 9.37 | 3.82 | 4.9 | 0.14 | 1.19 | 35.08 |
| 2008 | 0.35 | 0.36 | 0.93 | 4.34 | 4.5 | 4.59 | 3.26 | 2.16 | 1.8 | 2.43 | 1.79 | 1.37 | 27.88 |
| 2009 | 0.67 | 0.96 | 1.74 | 2.12 | 1.71 | 3.59 | 2.37 | 3.81 | 1.58 | 6.55 | 0.91 | 2.5 | 28.51 |
| 2010 | 0.8 | 0.9 | 1.51 | 1.94 | 2.53 | 7.59 | 5.72 | 3.08 | 10.38 | 1.14 | 1.94 | 2.51 | 40.04 |
| 2011 | 0.85 | 1.21 | 1.73 | 3.42 | 4.72 | 5.24 | 5.5 | 1.03 | 0.94 | 0.54 | 0.17 | 1.09 | 26.44 |
| 2012 | 0.62 | 2.12 | 1.49 | 3.27 | 7.45 | 3.34 | 1.91 | 2.09 | 0.78 | 1.25 | 0.61 | 1.41 | 26.34 |
| 2013 | 0.64 | 1.14 | 2.02 | 5.71 | 5.72 | 6.38 | 3.11 | 2.34 | 1.41 | 3 | 0.73 | 0.85 | 33.05 |
| 2014 | 0.74 | 0.93 | 0.87 | 5.2 | 2.39 | 10.4 | 1.29 | 4.18 | 2.26 | 1.46 | 0.81 | 0.91 | 31.44 |
| 2015 | 0.45 | 0.6 | 0.73 | 2.74 | 5.09 | 4.95 | 4.92 | 4.46 | 3.86 | 1.72 | 3.99 | 2.91 | 36.42 |
| 2016 | 0.39 | 0.7 | 2.36 | 2.7 | 4.36 | 4.64 | 7.5 | 7.01 | 8.27 | 3.98 | 1.47 | 1.6 | 44.98 |
| Monthly | 0.637 | 1.047 | 1.627 | 3.314 | 4.16 | 5.359 | 3.824 | 3.953 | 3.51 | 2.697 | 1.256 | 1.634 | 33.018 |
| avg, | | | | | | | | | | | | | |
| inches | | | | | | | | | | | | | |

Step 2: Calculate the % deviation of each year from the annual average. An example is shown below.

| | Annual PRCP, | |
|---|---|---|
| Year | inch | % Deviation |
| 2007 | 35.08 | 6.2 |
| 2008 | 27.88 | 15.6 |
| 2009 | 28.51 | 13.7 |
| 2010 | 40.04 | 21.3 |
| 2011 | 26.44 | 19.9 |
| 2012 | 26.34 | 20.2 |
| 2013 | 33.05 | 0.1 |
| 2014 | 31.44 | 4.8 |
| 2015 | 36.42 | 10.3 |
| 2016 | 44.98 | 36.2 |
| Avg. inch | 33.02 | |

Step 3: Select the three years with minimum deviation from the annual average. An example is shown below.

| | Annual PRCP, | |
|---|---|---|
| Year | inch | % Deviation |
| 2007 | 35.08 | 6.2 |
| 2013 | 33.05 | 0.1 |
| 2014 | 31.44 | 4.8 |

Step 4: Calculate the monthly deviations from the average monthly precipitation for each of the three years. An example is shown below. Note that in the last column, the percent deviation was divided by 12 to obtain an equivalent weight for the monthly distribution and the annual cumulative rainfall.

| | | | | | | | | | | | | | | % |
|---|---|---|---|---|---|---|---|---|---|---|---|---|---|---|
| | | | | | | | | | | | | | % | Dev./ |
| Year | January | February | March | April | May | June | July | August | September | October | November | December | Dev. | 12 |
| 2007 | 35.0 | 48.0 | 77.6 | 48.7 | 24.8 | 46.4 | 30.4 | 137.0 | 8.8 | 81.7 | 88.9 | 27.2 | 54.6 | 4.5 |
| 2013 | 0.5 | 8.9 | 24.2 | 72.3 | 37.5 | 19.1 | 18.7 | 40.8 | 59.8 | 11.2 | 41.9 | 48.0 | 31.9 | 2.7 |
| 2014 | 16.2 | 11.2 | 46.5 | 56.9 | 42.5 | 94.1 | 66.3 | 5.7 | 35.6 | 45.9 | 35.5 | 44.3 | 41.7 | 3.5 |

Step 5: Calculate the average of percent deviations from Step 3 and Step 4 for each of the three years. An example is shown below.

| | % Annual Deviation | % Monthly Deviation | |
|---|---|---|---|
| Year | (Step 3) | (Step 4) | Average |
| 2007 | 6.2 | 4.5 | 5.4 |
| 2013 | 0.1 | 2.7 | 1.4* |
| 2014 | 4.8 | 3.5 | 4.2 |

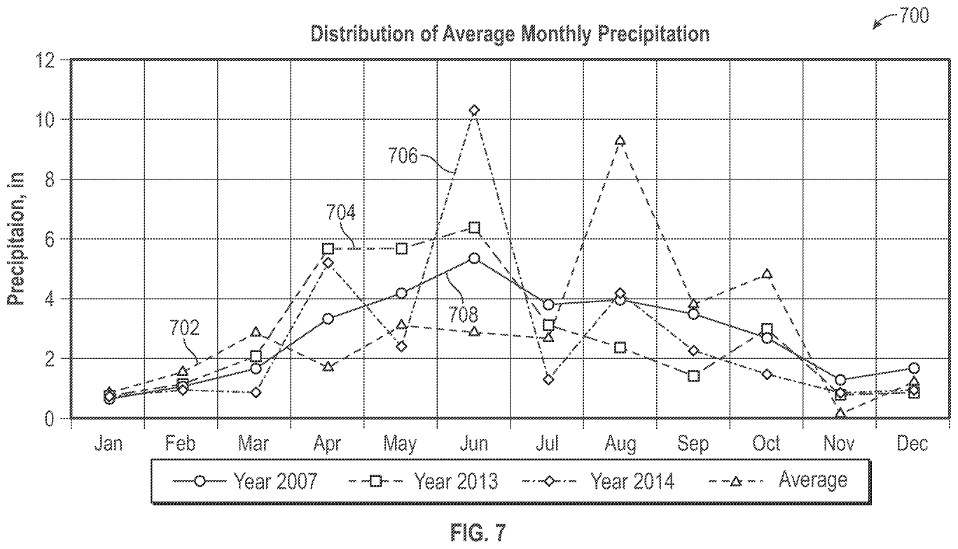

Conclusion: Year 2013* is considered a normal year and its daily weather data will be used as the best forecast of future weather. FIG. 7 shows an example of the distribution of average monthly precipitation 700. As shown in FIG. 7, there are three years present 702, 704, 706, as well as an average 708.

Variability of incoming solar radiation and its duration, in the form of direct radiation, is analyzed across the weather station network in North America throughout the year. This information guides the design of solar power components, such as battery, charge controller, and panel, for weather stations. It is assumed that calculations are performed with a south facing aspect and a clear sky. The solar radiation data is obtained from The Weather Company and directly used in the model.

Another factor that defines this invention is that calculations are done with both linear plateau and quadratic plateau equations. Quadratic equations are generally used by agronomists to estimate the economic maximum return to nitrogen. The curve maximum is calculated to provide the optimum rate; however, quadratic equations alone are usually insufficient. This new approach uses non-linear equations to look for the point at which a constant return for nitrogen fertilizer inputs is realized. This inflection point is where the return becomes flat and consistent. Quadratic plus plateau along with linear plateau equations are employed.

This disclosure takes a comprehensive approach, combining field-centric variables to provide accurate nitrogen recommendations specific to a field. There are a number of well-known, and publicly available, process-based models (e.g., APSIM, RZWQM, CropSyst, DSSAT, and SALUS), which could be utilized in this simulation. However, the primary advantage of this approach is that the crop model is exclusive in that it integrates genetic coefficients, soil data, management data, and current weather with linear plateau and quadratic plateau equations.

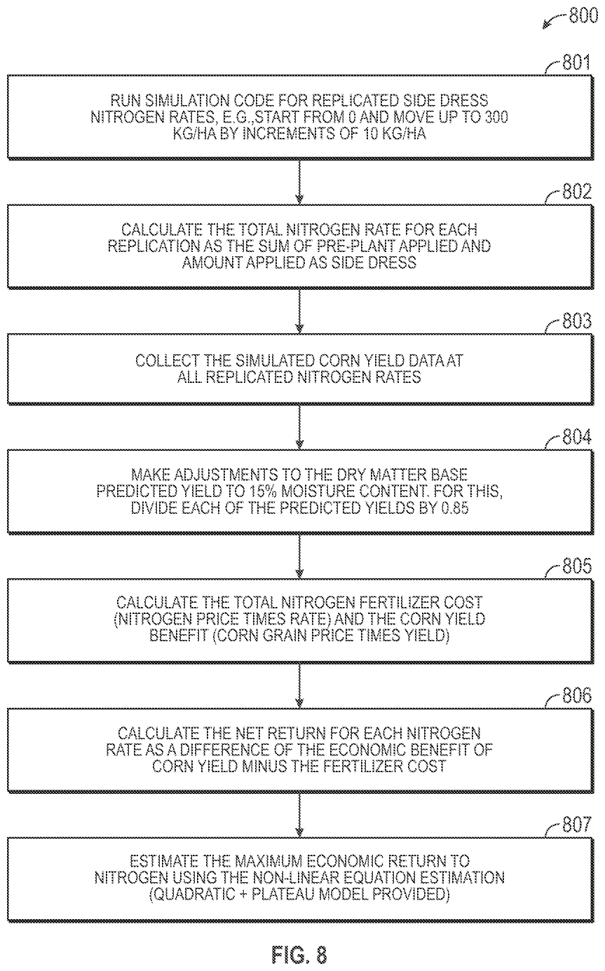

The process-based model simulates crop yields at various added nitrogen applications, and it is automatically updated daily. An example illustrating steps involved in the procedure is shown in FIG. 8. The methodology 800 shown in FIG. 8 is used for estimating effective crop nitrogen applications is outlined in the following sections.

At step 801, the methodology computes the replicated side-dress nitrogen rates. For example, the software may run from zero up to 300 kg/ha by a defined set of increments, e.g., 10 kg/ha.

At step 802, calculate the total nitrogen rates for each replication as the sum of pre-plant applied and the amount applied as side dress.

At step 803, collect the simulated corn yield data at all replicated nitrogen rates.

At step 804, make adjustments to the dry matter base predicted yield to 15% moisture content. For this, divide each of the predicted yields by 0.85.

At step 805, calculate the total nitrogen fertilizer cost (nitrogen price times rate) and the corn yield benefit (corn grain price times yield). The price of corn and nitrogen are two important inputs in the calculation that are obtained from the grower or the local agronomist.

At step 806, calculate the net return for each nitrogen rate as a difference of the economic benefit of corn yield minus the fertilizer cost.

At step 807, determine an estimate of the maximum economic return to nitrogen fertilizer using the non-linear equation estimation (quadratic-plateau model provided).

Linear Plateau Equation:

Y=a+b*X, if X<X0
Y=P, if X>=X0
Y=Return
X=Nitrogen Rate
X0=The critical point after which the increase of nitrogen fertilizer can no longer increase return
P=maximum return

Quadratic Plateau Equation:

Y=a+b*X+c*X\hat{ }2, if X<X0

Y=P, if X>=X0
Y=Return
X=Nitrogen Rate
X0=The critical point after which the increase of nitrogen fertilizer can no longer increase return
P=maximum return

There are limitations when using non-linear models to calculate a critical point. Non-linear methods rely on iterative procedures that require a speculative starting value. The procedure calculates sum-of-squares and automatically stops when there is not a significant decrease in the sum-of-squares with an additional iteration of starting values. A potential restriction is that the optimization may fail. If the simulated data points do not follow a linear-plateau or quadratic-plateau curve, the optimization algorithm to estimate X0 may have difficulty in determining the starting values. Also, if the simulation does not converge after 10,000 iterations, the optimization will fail.

With this method, an estimate of maximum economic return to nitrogen is calculated by fitting a regression model with crop yield and applied nitrogen as the variables. Pre-plant nitrogen levels are determined using simulated data and then nitrogen applications are optimized to reach yield targets and increase profitability. The grower is provided with side-dress nitrogen recommendations to zones within fields, as well as predicted yield, dates to the six-leaf growth stage, and the predicted major nitrogen balance components. All these items are presented to the grower through a user interface on a device.

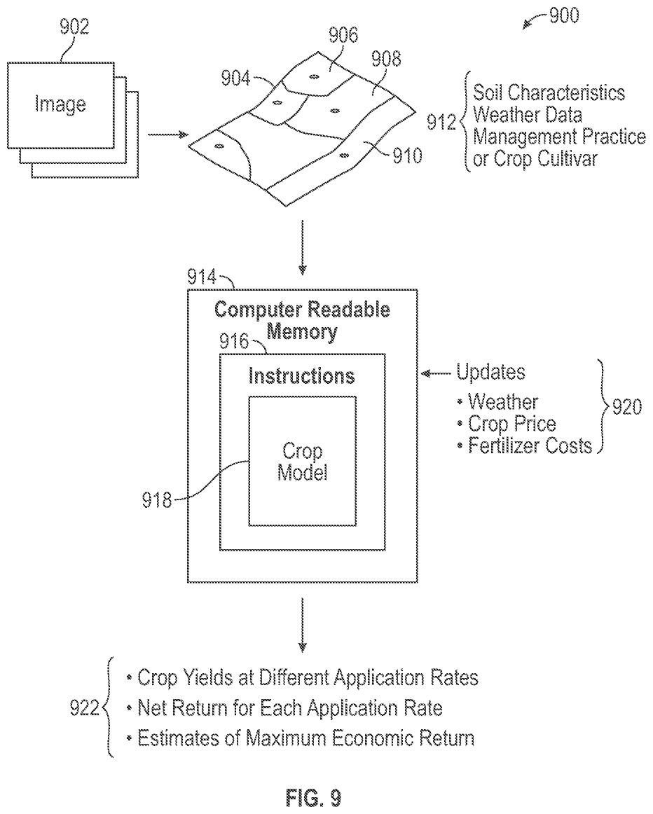

FIG. 9 illustrates another example 900. Imagery 902 is shown for an agricultural field. As previously explained, the imagery may be acquired through remote sensing from any number of types of platforms. The imagery may be used such as to determine vegetation indexes which may then me used to delineate the agricultural field into a plurality of management zones such as management zones 904, 906, 908, and 910 as shown. Additional inputs 912 may be provided for each of the management zones such as soil characteristics, weather data, management practices, and crop cultivar. Note that the soil characteristics may be determined from physical samples within each of the management zones. The management zones and input data 912 may be used by a simulation model or crop model 918. The crop model 918 may be implemented as a set of instructions 916 which may be stored on a non-transitory computer readable medium 914. During the growing season, as the model is applied, updates 920 of data may be applied; these may include updated weather information such as daily weather updates, updated crop process, and updated fertilizer costs. Results or output from the model may be provided in any number of different ways including on a screen display associated with a software application or web site, through emails, texts, or otherwise. The results 922 may include, without limitation, crop yields for each management zone at different application rates, net return for each application rate, and estimates of maximum economic return.

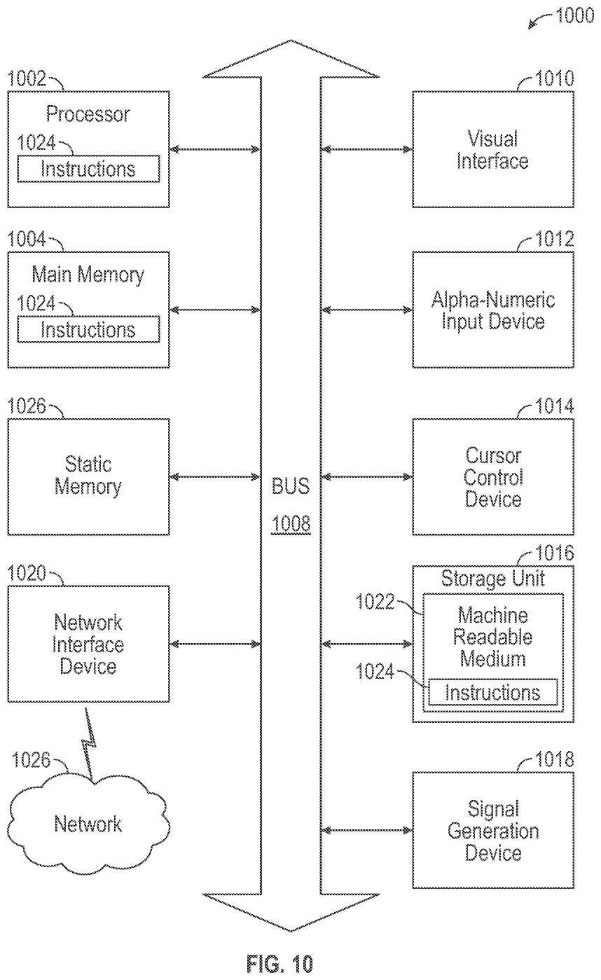

FIG. 10 is a block diagram illustrating components of an example machine for reading and executing instructions from a machine-readable medium which is one example of a computing environment. The computer system 1000 can be used to execute instructions 1024 (e.g., program code or software) for causing the machine to perform any one or more of the methodologies (or processes) described herein. In alternative embodiments, the machine operates as a standalone device or a connected (e.g., networked) device that connects to other machines. In a networked deployment, the machine may operate in the capacity of a server machine or a client machine in a server-client system environment 1000, or as a peer machine in a peer-to-peer (or distributed) system environment 1000.

The machine may be a server computer, a client computer, a personal computer (PC), a tablet PC, a set-top box (STB), a smartphone, an internet of things (IoT) appliance, a network router, switch or bridge, or any machine capable of executing instructions 1024 (sequential or otherwise) that specify actions to be taken by that machine. Further, while only a single machine is illustrated, the term “machine” shall also be taken to include any collection of machines that individually or jointly execute instructions 1024 to perform any one or more of the methodologies discussed herein.

The example computer system 1000 includes one or more processing units (generally processor 1002). The processor 1002 is, for example, a central processing unit (CPU), a graphics processing unit (GPU), a digital signal processor (DSP), a controller, a state machine, one or more application specific integrated circuits (ASICs), one or more radio-frequency integrated circuits (RFICs), or any combination of these. The computer system 1000 also includes a main memory 1004. The computer system may include a storage unit 1016. The processor 1002, memory 1004, and the storage unit 1016 communicate via a bus 1008.

In addition, the computer system 1000 can include a static memory 1006, a graphics display 1010 (e.g., to drive a plasma display panel (PDP), a liquid crystal display (LCD), or a projector). The computer system 1000 may also include an alphanumeric input device 1012 (e.g., a keyboard), a cursor control device 1014 (e.g., a mouse, a trackball, a joystick, a motion sensor, or other pointing instrument), a signal generation device 1018 (e.g., a speaker), and a network interface device 1020, which also are configured to communicate via the bus 1008.

The storage unit 1016 includes a machine-readable medium 1022 on which is stored instructions 1024 (e.g., software) embodying any one or more of the methodologies or functions described herein. For example, the instructions 1024 may include the functionalities of modules of the client device or network system. The instructions 1024 may also reside, completely or at least partially, within the main memory 1004 or within the processor 1002 (e.g., within a processor's cache memory) during execution thereof by the computer system 1000, the main memory 1004 and the processor 1002 also constituting machine-readable media. The instructions 1024 may be transmitted or received over a network 1026 via the network interface device 1020.

While machine-readable medium 1022 is shown in an example embodiment to be a single medium, the term “machine-readable medium” should be taken to include a single medium or multiple media (e.g., a centralized or distributed database, or associated caches and servers) able to store the instructions 1024. The term “machine-readable medium” shall also be taken to include any medium that is capable of storing instructions 1024 for execution by the machine and that cause the machine to perform any one or more of the methodologies disclosed herein. The term “machine-readable medium” includes, but is not be limited to, data repositories in the form of solid-state memories, optical media, and magnetic media.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the disclosure. This description should be read to include one or at least one and the singular also includes the plural unless it is obvious that it is meant otherwise.

Upon reading this disclosure, those of skill in the art will appreciate still additional alternative structural and functional designs for systems, methods, and apparatus for monitoring crop conditions within agricultural fields. For example, differences in the manner in which images are obtained are contemplated including satellite imagery, aerial imagery from drones, or other types of imagery. Variations in the type of computing environments used are fully contemplated. Variation in the manner in which soil samples are obtained or weather data is obtained, or other data acquisition variations is fully contemplated. Variations in the types of vegetation indices used are contemplated. Various steps described in processing are optional and need not necessarily be performed in a particular embodiment. Other variations are contemplated as may be appropriate based on a particular crop, particular geographic location of the field, available computing resources, or other factors. Thus, while particular embodiments and applications have been illustrated and described, it is to be understood that the disclosed embodiments are not limited to the precise methodologies disclosed herein. Various modifications, changes and variations, which will be apparent to those skilled in the art, may be made in the arrangement, operation and details of the method and apparatus disclosed herein without departing from the spirit and scope of the disclosure.

### REFERENCES

The following references are cited herein and hereby incorporated by reference in their entireties.
Hernandez, J. A., and Mulla, D. J., (2008). Estimating Uncertainty of Economically Optimum Fertilizer Rates. Agron. J. 100, 1221-1229. doi: 10.2134/agronj2007.0273
Puntel, L. A., Sawyer, J. E., Barker, D. W., Dietzel, R., Poffenbarger, H., Castellano, M. J., Moore, K. J., Thorburn, P., and Archontoulis, S. V. (2016). Modeling Long-Term Corn Yield Response to Nitrogen Rate and Crop Rotation. Front. Plant Sci. 7:1630. doi: 10.3389/fpls.2016.01630
Sawyer, J. E., (2015). Nitrogen Use in Iowa Corn Production. Iowa State University Extension and Outreach. Crop 3073

What is claimed is:

1. A method for effective nitrogen application, the method comprising:
acquiring imagery of an agricultural field;
delineating a plurality of management zones within the agricultural field using the imagery of the agricultural field;
for each of the management zones within the agricultural field receiving soil characteristics at a computing device, the soil characteristics derived from physical soil samples within the management zones;
for each of the management zones within the agricultural field receiving weather data at the computing device;
for each of the management zones receiving management practice information and crop cultivar identification at the computing device;
applying a crop model implemented by instructions stored on a computer readable medium and executed on the computing device to simulate effects of in-season nitrogen applications on crop yields within each of the management zones, wherein the crop model is parameterized with the soil characteristics, the weather data, the management practice information, and the crop cultivar identification in order to provide in-season nitrogen recommendations for the crop;
updating inputs to the crop model a plurality of times during the growing season.

2. The method of claim 1 wherein the updating the inputs to the crop model includes providing updated weather data to the crop model for the growing season.

3. The method of claim 1 wherein the updating the inputs to the crop model is performed on a periodic basis.

4. The method of claim 3 wherein the periodic basis is a daily basis.

5. The method of claim 1 wherein the soil characteristics include soil texture, organic matter, pH, cation exchange capacity (CEC), and soil nitrogen content.

6. The method of claim 1 wherein the crop model uses linear-plateau and quadratic-plateau equations to calculate effects of different nitrogen rates.

7. The method of claim 1 wherein the crop model simulates crop yields at a plurality of added nitrogen application rates.

8. The method of claim 1 wherein the crop model is further parameterized with a crop price.

9. The method of claim 1 wherein the crop model is further parameterized with a nitrogen fertilizer cost.

10. The method of claim 1 wherein the crop model determines a net return for each of a plurality of added nitrogen application rates.

11. The method of claim 1 wherein the crop model determines an estimate of a maximum economic return for nitrogen fertilizer using a quadratic-plateau model.

12. The method of claim 1 wherein the imagery of the agricultural field is used to determine a vegetation index and the vegetation index is used in delineating the plurality of management zones within the agricultural field.

13. A system for effective nitrogen application within an agricultural field during a growing season, the system comprising:
a computing environment including at least one computer-readable storage medium having program instructions stored therein and a computer processor operable to execute the program instructions to apply a crop model;
wherein the crop model simulates effects of in-season nitrogen applications on crop yields within each of a plurality of management zones within the agricultural field;
wherein the crop model is parameterized with soil characteristics obtained from soil samples within the plurality of management zones, weather data including weather data collected during the growing season, management practice information, and crop cultivar identification in order to provide in-season nitrogen recommendations for the crop.

14. The system of claim 13 wherein the soil characteristics include soil texture, organic matter, pH, cation exchange capacity (CEC), and soil nitrogen content.

15. The system of claim 13 wherein the crop model uses linear-plateau and quadratic-plateau equations to calculate effects of different nitrogen rates.

16. The system of claim 13 wherein the crop model simulates crop yields at a plurality of added nitrogen application rates.

17. The system of claim 13 wherein the crop model is further parameterized with a crop price.

18. The system of claim 17 wherein the crop model is further parameterized with a nitrogen fertilizer cost.

19. The system of claim 18 wherein the crop model determines a net return for each of a plurality of added nitrogen application rates.

20. The system of claim 18 wherein the crop model determines an estimate of a maximum economic return for nitrogen fertilizer using a quadratic-plateau model.
